(19) United States
(12) Patent Application Publication
(10) Pub. No.: US 2015/0314114 A1
(43) Pub. Date: Nov. 5, 2015

(54) COLLAGEN DEVICE

(71) Applicant: ITALIA MEDICA SRL, Milano (IT)

(72) Inventor: Antonio Luigi LA ROSA, Pavia (IT)

(21) Appl. No.: 14/650,744

(22) PCT Filed: Dec. 10, 2013
(86) PCT No.: PCT/IB2013/002718

§ 371 Date: Jun. 9, 2015

(30) Foreign Application Priority Data
Dec. 10, 2012 (IT) MI2012A002095

(51) Int. Cl. A61M 35/00; A61F 13/02; A61M 25/02; A61F 13/00

(52) U.S. Cl. CPC A61M 35/00; A61F 13/00012; A61F 13/0216; A61M 25/02; A61F 2013/0017; A61F 2013/00285; A61M 2025/0266

(57) ABSTRACT

The present invention relates to a device adapted for dressing and treating wounds, skin lesions/ulcers, sores and burns.

### STATE OF THE ART

The present invention is directed to a device adapted for dressing and treating wounds, skin lesions/ulcers, sores and burns.

As it is well known, wound healing involves the activation of a complex biological process aimed at filling the discontinuity represented by the wound with a permanent structure of connective nature, which is the scar. In practice, the healing process consists of a series of events leading to the new-formation of a tissue of connective nature, i.e. a tissue other than the original tissue, the events occurring in distinct phases which can be schematized as follows.

### Inflammatory Phase

In the case of wound, ulcer or sore, the inflammatory phase acts for circumscribing and eliminating the microbial agent, any foreign body present in the wound, and necrotic cells. Furthermore, during this phase, factors are activated which constitute the basis for subsequent proliferative processes, thereby being required to repair or replace the damaged tissue.

This phase involves vasodilatation, plasma exudation and proliferation of macrophages, i.e. mononuclear cells with phagocytic capabilities which cooperate with neutrophil granulocytes to cleanse the wound. Once the wound is clean, the macrophages produce growth factors which allow for proceeding to the next phase, which is referred to as the proliferative phase or proliferation phase.

### Proliferative phase

The collagen damaged and fragmented by the proteolytic enzymes released by macrophages leads to a further chemotactic recruitment of macrophages and fibroblasts into the wound. The production of collagen by fibroblasts recruited into the wound as well as the angiogenesis induced by growth factors trigger the activation of the proliferative phase, with formation of granulation tissue. The granulation tissue, which is fragile and bleeds easily, is formed starting from the bottom towards the surface of the wound, and it is able to fill large areas of loss of substance. During this phase, cell clusters are formed which are welded to each other following the scaffold formed by the fibrin network, thereby resulting in generation of blood vessels and formation of a new vasculature. The proliferation of fibroblasts at the site of the ulcer leads to the production of collagen fibers (predominantly type III collagen or transitional collagen), a fibrous mucopolysaccharide-like protein replacing the fibrin and representing the main protein of connective tissue in animals, to form the extracellular matrix (ECM). There are numerous types of collagen, but the most relevant are those of types I, II and III. Type I collagen represents 90% of total collagen in humans, and it contributes to composition of major connective tissues such as skin, bones, tendons and cornea. Conversely, type II collagen forms cartilage, intervertebral discs and vitreous body. Type III collagen, or transitional collagen, is of great importance in the cardiovascular system, and it is the collagen involved in the repair process, i.e. the granulation process. Fibroblasts remain active for the time required for the collagen product to successfully fill the wound and, when their task is fulfilled, they disappear. Once the loss of substance has been settled, the epithelial cells advance from the edges of the lesion to cover the granulation tissue with a thin layer of epidermis. Generally, in case of small lesions and in absence of factors which could hinder healing, the time required to obtain the closure of a wound by re-epithelialization is in the range from 24 to 30 days.

### Remodelling phase

This phase is characterized by the definitive, stable closure of the wound with a scar. Type III collagen is degraded by fibroblasts and gradually replaced by type I collagen or permanent collagen. The complete replacement of the transitional extracellular membrane with type I collagen requires a time of more than six months in order to obtain the formation of a tissue having 80% of the strength of the original tissue.

However, the wound- and lesion-healing process can be adversely affected by certain factors occurring both locally and systemically. Some of the factors which can adversely affect the healing of wounds are, for example, an altered blood supply due to the presence of vascular-type diseases, the release of proteolytic enzymes accompanying ischemic and necrotic tissues, local diseases such as eczema, the onset of infections causing the tissue to reside in a long-lasting inflammatory state, the age of the subject, the presence of any systemic disease such as, particularly, diabetes. When the healing process described above is influenced by factors impairing a smooth development thereof, it can be said that ulcers—rather than wounds—are present.

Particularly, in case of ulcers (seen as wounds for which the regular healing process is not feasible due to factors such as, for example, those described above) on lower limbs, the incidence of vascular causes leading to healing failure is about 90%. The cause for this impediment in the healing process is due to a vascular impairment of not only the major vessels but also the minor vessels and, therefore, it is obvious that the incidence of this phenomenon is quite high.

In the presence of ulcers, their cleansing is often difficult and painful, and they require long-time treatments in order to be able to achieve a condition which is suitable for stimulating granulation or forming a dermal structure.

The presence of necrotic tissue as well as of fibrin, eschar and infection prevents the tissue from re-growing, and this is the reason why the removal thereof should occur as soon as possible and possibly radically. Generally, this removal is performed either mechanically or pharmacologically, with the latter case which requires 1-2 months on average.

Also in case, for example, of chronic ulcers, the granulation process is particularly difficult to be triggered due to the presence of a high number of neutrophils and macrophages producing high amounts of inflammatory mediators and proteolytic enzymes. In fact, inflammatory mediators cause the tissue to be degraded and the formation of extracellular matrix to be inhibited. Chronic ulcers are frequently characterized by the presence of bacterial colonies as well as by the presence of chronic inflammatory states and degradation of the extracellular matrix (ECM), leading to senescence of fibroblasts and degradation and inactivation of growth factors. The occurrence of bacterial infections is promoted by the presence of necrotic tissue and fibrin. Infections compete and interfere with tissue growth processes by damaging the cells through oxygen subtraction and toxin release mechanisms. The presence of infections causes a blockade in the tissue repair processes, an increase of the ulcer in size, as well as an increased pain accompanied by characteristic “burning” symptoms. Generally, the most suitable treatment to remedy this kind of complications involves the parenteral administration of antibiotics. In many cases of chronic wounds, however, resistant bacteria are found which induce chronic inflammatory states retarding the healing process, and which are poorly sensitive to normal antimicrobial therapies. When the systemic therapy is not particularly effective despite the use of all the proper precautions and the correct application of the existing protocols for the given case, a topical therapy based on antibiotic products might give good results. However, such a therapy is difficult to be implemented because it is very difficult to deliver the desired drug to a site, but especially to maintain it at the site for a time long enough to allow the drug to exert its action at best. Furthermore, particularly in case of ulcers, but generally in all cases in which there are wounds and lesions of various nature, the presence of painful states is significant and often persistent not only when the wounds, lesions or ulcers themselves are mechanically cleaned, but also on different occasions, due to the existence of inflammatory and infectious phenomena. These painful states are normally treated by the enteral or parenteral administration of drugs. In case of acute pain syndromes, a pharmacological treatment with opioids such as codeine, oxycodone or tramadol can be used. Also in this case, the enteral or parenteral administration of opioid drugs results in a number of significant side effects, and it should therefore be avoided or at least drastically limited. Accordingly, it is necessary to identify alternative administration modes other than a systemic administration for the administration of analgesically active drugs in order to counteract the side effects of the most effective painkillers commonly used (for example, opioids) while ensuring the same efficacy. For example, an effective local administration of lidocaine could easily eliminate the painful condition without causing any adverse side effects on ulcer healing or bacterial proliferation, as well as without causing any of the adverse side effects typically experienced by the entire body when opioids are administered systemically.

Catheters adapted for delivery of, for example, an antimicrobial agent to a site, which can be either a damaged area or an open wound, are known from the prior art. These catheters, such as those described in US 2006/0135941, are intended for intra-arterial/intra-venous insertion, and they comprise a tubular body optionally provided with multiple holes and optionally wrapped by a membrane. Collagen membranes for the treatment of wounds are also known, for example, from WO 2006/002326. These membranes are soaked with therapeutically effective substances, such as substances in slow-release formulations.

### OBJECTS OF THE INVENTION

An aim of the present invention is to provide a device adapted for dressing and treating wounds, skin lesions/ulcers, sores and burns, which can establish and maintain a direct and sustained contact of them with one or more therapeutically useful substances.

Another aim of the present invention is to provide a device adapted for dressing and treating wounds, skin lesions/ulcers, sores and burns, which allows them to be further treated at home without requiring the intervention of qualified personnel.

A further aim of the present invention is to provide a device adapted for dressing and treating wounds, skin lesions/ulcers, sores and burns, which allows said dressing to be effectively maintained for a time significantly greater than that achieved with a similar dressing according to the known art.

Still another aim of the present invention is to provide a device adapted for dressing and treating wounds, skin lesions/ulcers, sores and burns, which allows for a sustained topical release of employed substances without any significant systemic side effects.

### DESCRIPTION OF THE INVENTION

These and still other objects and related advantages, which will be better clarified by the following description, are achieved by means of a device adapted for dressing and treating wounds, skin lesions/ulcers, sores and burns, which comprises:
a) at least one biocompatible and optionally reabsorbable membrane
b) at least one catheter coupled to said membrane.

According to the present invention, the term “biocompatible membrane” or “bio-membrane” means a membrane made of a natural or synthetic material which is used either to replace part of a living system or to operate in close contact with tissues of a living organism, which can be well tolerated by the host organism where it is intended to act, and which can elicit a proper response depending on the application thereof.

Still according to the invention, the term “reabsorbable material” means a material which is able to be chemically degraded and controllably reabsorbed within a living system so as to be replaced by the host tissue.

According to the present invention, said biocompatible and optionally reabsorbable membrane can advantageously be made of a natural or synthetic material. In the first case, a particularly suitable material is collagen, that is a fibrous mucopolysaccharide-like protein constituting the essential unity of the intercellular substance of connective tissue and mainly comprising glycine, proline and hydroxyproline. In case of biocompatible reabsorbable membranes of synthetic type, an example can be a membrane made of a polymeric material which can be “biologically inert” or else “degradable”. Biologically inert materials include PMMA (polymethylmethacrylate), PDMS (polydimethylsiloxane) and UHMWPE (ultra high molecular weight polyethylene). A polymer is degradable if the degradation products thereof are also biocompatible. One of these is polylactic acid, which is increasingly employed in tissue engineering. The degradation of a polymer can occur (i) by water absorption followed by hydrolysis of unstable bonds (hydrolytic degradation), or (ii) by enzymes or micro-organisms (biodegradation).

Said membrane can be of various sizes and shapes (e.g., square, rectangular, circular or elliptical), depending on the morphological features of the lesion to be treated. Still according to the present invention, the membrane can be made of a non-reabsorbable material and, in this case, it will be either periodically replaced or permanently removed depending on the wound to be treated and the expected healing times.

Still according to the present invention, said membrane can have windows of various shapes (for example, circular, diamond-shaped, triangular, hexagonal, etc.) and sizes on its surface. For example, in case of a membrane of 3.5 cm×3.5 cm in size, said windows may have a circular shape with a diameter of between about 1 mm and about 6 mm. When the above windows are present and, for example, if they are circular in shape, they may be the same or different from each other in diameter, and they may be variously distributed on the surface of the membrane itself.

The catheter is flexible and preferably shaped as a tube, i.e. able to administrate a given compound, and it is preferably made of any soft, biocompatible, hypo-allergenic material. It is made in various lengths and diameters according to the needs, which depend on the type of lesion to which the device according to the invention is intended to be applied and the type of solution to be infused at low or high density. Said catheter may be single or multiple, and it is firmly coupled to the biocompatible membrane.

The device according to the invention is also provided with:
c) means adapted for allowing the device to adhere to the site of the wound, lesion, ulcer, sore or burn.

Said adhesion-oriented means of the device are constituted, for example, by adhesive patches which can be placed to partially or completely cover the membrane once it has been positioned on the wound to be treated.

Particularly, according to a preferred embodiment of the present invention, the biocompatible membrane is of a slow, full resorption type, and it acts to induce and maintain the granulation process, i.e. the tissue regrowth process at the site of the lesion.

Practically, the device according to the invention is advantageously used for dressing and treating wounds, skin lesions/ulcers, sores and burns by positioning the biocompatible membrane on the wound or lesion to be treated, and securing it in place with the use of the adhesion-oriented means of the device. One end of the catheter will be firmly coupled to and in contact with said membrane, whereas the other end will be free and connectable to a suitable system for the administration of a liquid, a suspension or other substance. Said liquid, suspension or other substance can advantageously be either normal saline or a mixture of saline and at least one pharmacologically active compound or pharmacologically active composition. Said liquid or suspension can then reach the membrane at the desired speed, and the membrane will be soaked therewith.

Accordingly, the device performs a dual function: the biocompatible material membrane acts for triggering or accelerating the granulation process and the subsequent healing of the wound or lesion, whereas the catheter allows for the simultaneous or sequential administration of one or more local and topical drugs which can be antibiotics/painkillers/anti-inflammatory agents/growth factors or other drugs, as required for the treatment of the lesion.

In fact, the device according to the present invention allows for the administration of different therapeutically effective substances as needed, without requiring removing the dressing whenever the ongoing therapy has to be changed. For example, in case of ulcers and open wounds, it is often required to evaluate the potential bacterial load existing in situ. Indeed, often a few days from the first dressing, there may be cases in which the bacteria initially present are replaced by, or associated with, other bacteria which require the use of antibiotics of a different type. In these cases, when the wounds are treated with membranes according to the known art, it is necessary to replace the dressing and administer—even more times a day—the proper topical antibiotic which can then be different from that used initially. In the same situation, the use of the devices according to the present invention allows either two different antibiotics to be sequentially administered via the catheter coupled to the membrane, or a new antibiotic other than that previously used to be administered without having to remove the membrane, and therefore without causing additional trauma and superinfection.

Accordingly, the device according to the present invention allows for “correcting”! “tuning” the ongoing therapy depending on changing conditions of the wound and/or of patient himself/herself, and without having to intervene continuously either on the affected area or on the dressing already made and properly positioned.

When saline alone is passed through the catheter, the membrane will be constantly moist yet allowing the wound to be maintained cleaned and washed.

In case of pharmacological administration of one or more active ingredients via the catheter, the membrane will be constantly soaked therewith and, therefore, it will actually constitute a slow-release topical administration system for the active ingredients suitable for the circumstance, thus allowing their action to be extended over time while assuring a topical and local administration thereof. At the same time, in virtue of its composition, it can accelerate the natural healing process of the wound.

As already stated, features of the membrane of the slow, full resorption type allow the granulation process and the tissue regrowth process to be induced and maintained at the site of the lesion.

The device of the invention comprises one, two or more superimposed membranes which can be of any shape, size and thickness, depending on the characteristics of the lesion to be treated.

Thus, the device of the invention is characterized by the ability of injecting, through the tube-like catheter provided therein, any therapeutically useful medicinal substance which will soak the membrane to be gradually diffused through the same to the underlying pathological tissues. In this case, the membrane acts for slowly delivering the drug introduced thereto, resulting in a “retard”-type sustained action of the drug over time.

As already said above, the membrane can have holes of various diameters and variously distributed on its surface to allow an optional drug, used in combination with the membrane alone, to be directly passed from the catheter to the underlying tissue in such a way as to obtain, in addition to a slow-release action, also an immediate action on the tissue to be treated.

The device is useful for making “retard” dressings, i.e. dressings depending on the resorption time of the used membrane, for the treatment of ulcers, sores and wounds in general. The membrane is either natural (e.g., collagen) or synthetic in nature, it is biocompatible, and its thickness and width can be varied according to the size of the wound. Moreover, the membrane is of a slow, full resorption type, and it is secured to the site of the lesion with the use of a suitable fastening system, for example an adhesive film. This device has the unique feature of performing a number of important functions related to the lesion repair process, thereby bringing significant advantages over the treatment systems which have been adopted so far.

Firstly, when the membrane is brought into contact with the lesion, it stimulates a chemotactic recruitment of fibroblasts from surrounding tissues, thereby triggering and maintaining active the granulation process to cause the growth of new tissue in the lesion and then the healing of the wound.

Furthermore, since said membrane is provided with a soft, flexible, biocompatible catheter which is firmly coupled thereto, it allows not only a qualified medical professional but also the patient himself/herself, to carry out the prescribed therapies even at home, through the infusion of drugs directly into the site of the lesion.

The use of infusion via catheter allows a local therapy to be performed, for example by soaking the membrane with antibiotics at set times in case of infection of the wound, and performing an antisepsis procedure on the wound several times a day, without removal of the dressing itself

In fact, in virtue of reabsorption characteristics of the membrane and the use of the catheter, the dressings made with the device according to the invention can be maintained in place for a time in the range of about 7-15 days, compared to the current 1-3 days expected for conventional dressings made according to the known art.

Furthermore, since the device of the invention is provided with a catheter, it also allows for an effective and sustained control of pain over time through the use of local painkillers. This aspect represents an enormous advantage over other options available until now, because it can eliminate and avoid the systemic use of painkillers which are often gastrolesive or otherwise subject to wide contraindications, such as in the case of morphine, for example.

Furthermore, the catheter is a useful means to pharmacologically interact with the lesion through the use of any drug which, in addition to antibiotics and painkillers, is believed to be useful for the healing of the lesion itself (for example, growth factors, liquid collagen, hyaluronic acid, steroidal and non-steroidal anti-inflammatory drugs). Accordingly, the features of the membrane allow for a sustained delivery of drugs which are optionally administered by means of the catheter coupled to the membrane itself, thus being able to achieve an optimal antibiotic and analgesic coverage free of side effects which are, instead, typical of the same drugs when administered systematically.

In addition, the presence of optional windows on the surface of the membrane allows for the direct transfer of the drug from the catheter to the underlying tissue, thereby resulting in a prompt and immediate action of the drug which is very important whenever a prompt analgesia is needed. At the same time, since the membrane is soaked with the drug itself, it is also possible to obtain a sustained action of the drug over time starting from a single administration.

In virtue of the above described features, the device according to the present invention can maintain an optimal micro-environment for about 7-15 days, thereby limiting repeated and frequent manipulations of the wounds and ulcers, which are often traumatic as well as prone to a high risk of bacterial pollution.

The following is a brief description of an experimental protocol used for the treatment of 286 patients with 312 chronic ulcers of the lower limbs. These patients were given previous treatments for the same disease according to the known art, for a period of time in the range from 3 months to 5 years, without achieving significant healing results for the wounds and lesions to be treated.

Of the 312 ulcers of the lower limbs under treatment, 252 were characterized by chronic venous insufficiency, 25 were of a mixed type, 21 were caused by vasculitis, 6 were caused by hypertension, and 8 were of traumatic origin. 146 ulcers were clinically infected. The size of the ulcers was between 5 cm² and 320 cm². The previous treatments according to the prior art involved a mechanical or enzymatic removal of the damaged tissue, a disinfection of the wound, and the dressing thereof. Antibiotic treatment was always given systemically.

The wounds were treated with the device according to the present invention, then an antibiotic therapy (Ceftriaxone 1 g) was administered by perfusion of the antibiotic solution, repeated every 12 hours, with the use of the catheter of the device of the invention, both for preventive purposes (7 days) and for therapeutic purposes, for longer periods, up to a complete remission. The catheter of the device according to the invention was also used to infuse a solution of 2% lidocaine for analgesic purposes. The membrane of the device of the invention can be either of reabsorbable type or non-reabsorbable type and, in the latter case, it is required to be removed and replaced after a certain time (7 days).

Therefore, the device according to the invention has been shown to induce rapid changes in morphological features of the ulcers. The bed of the ulcers remained clean, the degree of inflammation existing around the ulcers was decreased very rapidly, and even the irregularity of the outer edges of the ulcers was reduced quickly. When the 146 clinically infected ulcers were treated with combined applications of antibiotic therapy and analgesic therapy, they showed a complete remission of the infection within 18-45 days. In no case there was a local irritation due to the application of the device according to the invention.

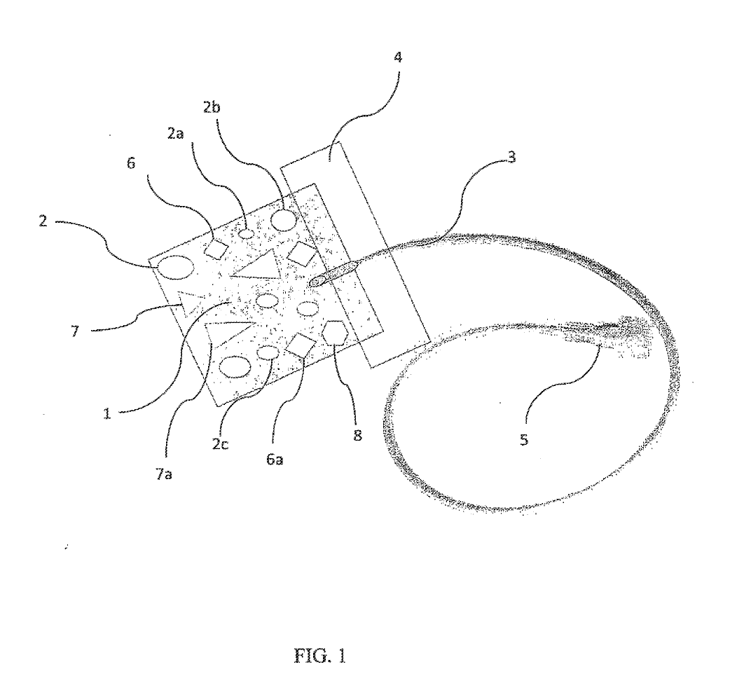

The device of the present invention is schematized as shown in the accompanying FIG. 1, given only by way of illustration and not by way of limitation, in which the membrane 1, made of a single sheet of a natural biocompatible material, preferably collagen, shaped as a square and provided with differently-sized windows or holes 2, 2a, 2b, 2c, 6, 6a, 7, 7a, 8, etc. which are randomly and evenly arranged on its surface, is firmly coupled to the catheter 3, which is made of a flexible material, by means of an adhesive patch 4. One end of the catheter 3 has a connector 5 adapted to be coupled to a differently-designed container for the administration of fluids or suspensions.

Still according to the present invention, the above described device in one of its embodiments may be sold as a ready-to-use kit and, in this case, it will comprise at least one biocompatible reabsorbable membrane and at least one catheter coupled to said membrane. Alternatively, the same device may be sold as a kit to be assembled, i.e., in the latter case, the kit will comprise at least one biocompatible reabsorbable membrane and at least one catheter adapted to be coupled to said membrane by a trained healthcare professional at the time of use.

1. Device adapted for dressing and treating wounds, skin lesions/ulcers, sores and burns, comprising:
a) at least one biocompatible membrane
b) at least one catheter coupled to said membrane.

2. Device according to claim 1, wherein said biocompatible membrane is reabsorbable.

3. Device according to claim 1, wherein said membrane is made of natural or synthetic material.

4. Device according to claim 3, wherein said material is collagen.

5. Device according to claim 1, wherein the surface of said membrane has one or more differently-sized windows or holes which are variously distributed over the surface of the membrane.

6. Device according to claim 1, further comprising two or more of said membranes in a superimposed condition.

7. Device according to claim 1, further comprising:
c) means adapted for allowing the device to adhere to the site of the wound, lesion, ulcer, sore or burn.

8. Device according to claim 1, wherein said membrane is of a slow, full resorption type.

9. Device according to claim 1, wherein one end of said catheter is firmly coupled to said membrane and in contact therewith, and the other end is free and connectable to a suitable system for the administration of a liquid or suspension.

10. Device according to claim 9, wherein said liquid or suspension consists of either a neat saline, or a mixture of saline and at least one pharmacologically active compound, or a pharmacologically active composition alone.

11. Device according to claim 10, wherein said pharmacologically active compound or pharmacologically active composition is selected from the group consisting of antibiotics, pain-killers, growth factors, liquid collagen, hyaluronic acid, steroidal and non-steroidal anti-inflammatory drugs.

12. Kit adapted for dressing and treating wounds, skin lesions/ulcers, sores and burns, comprising at least one biocompatible reabsorbable membrane and at least one catheter adapted to be coupled to the membrane at the time of use.

13. Use of the device according to claim 1 for dressing and treating wounds, lesions, ulcers, skin ulcers, sores and burns.

14. Use of the device according to claim 1 for producing “retard” dressings for wounds, lesions, ulcers, skin ulcers, sores and burns.
